(19) United States
(12) Patent Application Publication
(10) Pub. No.: US 2009/0093737 A1
(43) Pub. Date: Apr. 9, 2009

(54) ULTRASOUND APPARATUS WITH TREATMENT LENS

(75) Inventors: James E. Chomas, San Carlos, CA (US); Timothy L. Proulx, Santa Cruz, CA (US); Adnan I. Merchant, Fremont, CA (US)

Correspondence Address: FULWIDER PATTON - CABOCHON AESTHETICS, 6060 CENTER DRIVE, TENTH FLOOR, LOS ANGELES, CA, 90045, US

(73) Assignee: CABOCHON AESTHETICS, INC., Menlo Park, CA (US)

(21) Appl. No.: 12/247,853

(22) Filed: Oct. 8, 2008

Related U.S. Application Data
(60) Provisional application No. 60/978,607, filed on Oct. 9, 2007.

(51) Int. Cl. A61N 7/00

(52) U.S. Cl. 601/2

(57) ABSTRACT

An ultrasound apparatus comprising an ultrasound transducer having a geometric focus; an acoustic lens assembly acoustically coupled to the ultrasound transducer; wherein the acoustic lens assembly includes a focal layer that serves to increase relative acoustic pressure in a treatment region proximal to the transducer's geometric focus.

### CROSS-REFERENCES TO RELATED APPLICATIONS

This application claims priority from U.S. Provisional Application No. 60/978,607, filed Oct. 9, 2007 incorporated by reference in its entirety.

### FIELD OF THE INVENTION

The invention generally relates to an ultrasound apparatus used to provide ultrasound treatment. More particularly, the invention relates to an ultrasound apparatus having a treatment lens that is designed to achieve high acoustic pressure in a desired treatment zone or zones while limiting pressure outside the desired regions.

### BACKGROUND

Ultrasound is used routinely for wide-ranging therapeutic applications, ranging from warming superficial and deep tissue by delivery of low intensity doses to high intensity focused ultrasound (HIFU) for tissue and tumor ablation or lithotripsy. High intensity ultrasound has recently been used to treat subcutaneous tissue for adipose reduction.

The demands of ultrasound treatment devices are significantly different from imaging devices due to their use of generally higher pressure or higher energy ultrasound. One of the concerns associated with using high pressure ultrasound is delivering sufficient pressure to the treatment zone without causing deleterious effects outside of the treatment zone.

Therapeutic ultrasound transducers can be either unfocused or focused by mechanical or electronic means. Unfocused transducers employ a flat ultrasonic resonator (e.g. piezoelectric ceramic) that is coupled directly to the body or by means of a flat matching/wear layer that provides effective acoustic coupling between the resonator and the tissue. The sound intensity field in the superficial (near field) region of a flat emitter is roughly equal in extent to the size of the aperture, so these transducers are generally used to deliver energy over a large treatment area. Without the benefit of focusing gain, emitted acoustic pressure for a flat transducer is limited by transducer efficiency and drive electronics, but can reach levels higher than diagnostic limits. Importantly, even an unfocused transducer exhibits effective “focusing” in the acoustic field due to diffraction effects, which produce pressures at the near field/far field boundary that can be comparable or higher than those in the near field region. The depth of this “natural” focus of a flat resonating aperture is proportional to the square of the physical extent of the aperture and inversely proportional to sound wavelength. This can place the high pressure zone of the natural focus in an undesirable region of the anatomy.

Focused transducers may be mechanically focused by means of physical curvature (e.g. spherical or cylindrical) of the emitting aperture or by use of a lens with a sound speed different than the propagating medium. Electronic focusing can be achieved by applying electrical excitation to each element in the transducer with a defined delay. Many therapeutic transducers are comprised of a single emitter, and thus focusing is generally achieved by mechanical means. In contrast to the unfocused case, focusing the sound beam amplifies the emitted pressure at the face of the transducer by a factor termed the focal gain. Focal gains of up to 20 or higher can be obtained with spherically focused transducers, resulting in substantial and potentially dangerous pressures in the field. Since focal gain is inversely proportional to beam width, the treatment area also diminishes with increasing focal gain.

Current devices and methods for ultrasound treatment have been primarily concerned with very deep and focused treatment regions. There is a need in the art for a device and method which enables treatment of a wider and shallower (subcutaneous) treatment area. Accordingly, an acoustic lens may be employed with either focused or unfocused ultrasound therapy transducers to achieve one or more of the followings objectives: (a) increase the acoustic intensity in the desired treatment region, (b) increase the size of the treatment area or (c) modify the sound intensity pattern within the treatment region, all while reducing the sound intensity in regions outside the treatment region to acceptable levels.

### SUMMARY OF THE INVENTION

Disclosed is an ultrasound apparatus including an ultrasound transducer having a geometric focus Fₜᵣₐₙₛ, the transducer producing ultrasound waves. The apparatus further includes an acoustic lens assembly including a focal layer acoustically coupled to the ultrasound transducer such that the ultrasound waves are directed through the focal layer. The focal layer includes at least two minor lenses, each minor lens having a natural geometric focus Fₘᵢₙₒᵣ with each the minor lens yielding a discrete treatment region such that the focal layer includes plural discrete treatment regions.

According to one aspect of the invention the ultrasound transducer produces ultrasound having a mechanical index below the cavitation threshold of tissue and below the threshold at which tissue will emulsify. Stated in other terms, the transducer produces ultrasound having a mechanical index between 1.9 and 2.5. Each of the minor lens increases the mechanical index within the treatment region to between 2 and 8.

According to another aspect of the invention the apparatus further includes a rotational drive mechanism operably attached to the lens assembly, and the drive mechanism rotationally driving the lens assembly such that the lens assembly rotates relative to the transducer.

According to another aspect of invention, the acoustic lens assembly includes a receptacle for receiving tissue to be treated, the receptacle bounded on top and encircled by downwardly depending sidewalls, the transducer directing ultrasound into the receptacle. The focal layer may form the top wall of the receptacle, or the downwardly depending sidewalls of the receptacle.

The apparatus may further include an ultrasound reflector operably connected to the downwardly depending sidewalls such that the reflector generally faces the ultrasound transducer, whereby the reflector reflects ultrasound back into the receptacle.

According to another aspect of invention, the ultrasound transducer includes a plurality of ultrasound transducers surrounding the receptacle. Moreover, the apparatus may further include a plurality of ultrasound reflectors operably connected to the downwardly depending sidewalls such that each reflector generally faces an ultrasound transducer, whereby the reflectors reflects ultrasound back into the receptacle.

Also disclosed is an ultrasound apparatus including an ultrasound transducer having a geometric focus Fₜᵣₐₙₛ, the transducer producing ultrasound waves. An acoustic lens assembly including a focal layer is acoustically coupled to the ultrasound transducer such that the ultrasound waves are directed through the focal layer. The acoustic lens assembly defining a receptacle for receiving tissue to be treated, the receptacle bounded on top and encircled by downwardly depending sidewalls, the transducer directing ultrasound into the receptacle. The acoustic lens assembly may include a vacuum port or a needle access port. The focal layer may form either the top wall or the downwardly depending sidewalls of the receptacle.

### BRIEF DESCRIPTION OF THE DRAWINGS

FIG. 1 is a sectional view of a treatment device including an ultrasound transducer and an acoustic lens having multiple focal zones;

FIG. 2 is a sectional view of a treatment device including an ultrasound transducer and an acoustic diverging lens having multiple focal zones;

FIG. 3A depicts a simulation of on-axis (axis normal to the center of the transducer face) sound pressure for a flat 500 kHz transducer of 1 inch diameter of the type depicted in FIG. 1;

FIG. 3B depicts a simulation of the same transducer used in the simulation in FIG. 3A with a spherical 20 mm (diverging) lens of the type depicted in FIG. 2;

FIGS. 4 and 5 illustrate a treatment device including a mechanically focused transducer that incorporates an acoustic lens with at least one minor lens located at some distance from the emitting face;

FIG. 6A is a functional block diagram and FIG. 6B is a bottom view of an ultrasound treatment device including an acoustic lens assembly rotationally coupled to an ultrasound transducer, and FIG. 6C shows the arc-like treatment zones produced by rotating the lens assembly;

FIGS. 7A and 7B are sectional views of a tissue treatment apparatus including a lens assembly and an ultrasound transducer assembly;

FIGS. 8A and 8B are sectional views of a ultrasound treatment device including a side-firing transducer;

FIGS. 9A and 9B are sectional views of a ultrasound treatment device including plural side-firing ultrasound transducers;

FIGS. 10A and 10B are sectional views of a ultrasound treatment device including plural side-firing ultrasound transducers each provided with at least two minor lens;

FIGS. 11A and 11B are view of an acoustic lens including plural concave minor lenses with a generally flat major lens;

FIGS. 12A and 12B are view of an acoustic lens including plural concave minor lenses with a generally convex major lens having Fresnel features;

FIGS. 13A and 13B are view of an acoustic lens assembly including plural concave minor lenses provided on a Fresnel type major lens.

### DETAILED DESCRIPTION OF THE PREFERRED EMBODIMENTS

Sound waves are focused or defocused by propagation from a medium of one speed of sound (c₁) to a medium with a different speed of sound (c₂).

In addition to having a sound speed different from tissue, a lens suitable for use with ultrasound requires low sound attenuation and acoustic impedance that is comparable to that of tissue to maximize transmitted power. Acoustic impedance is a function of sound speed and apparent density of the material. For tissue, with a density of 1 g/cm³ and a sound speed of approximately 1520 m/s, acoustic impedance is approximately 1.52 MRayl. Several polymers, including a low density polyethylene, such as LDPE-4012 from Dow Plastics or polymethylpentene thermoplastics such as TPX MX002 or TPX DX845 from Mitsui Plastics are suitable. These materials have acoustic impedances between 1.5 and 1.8 MRayl and sound speeds in the range of 1900-2200 m/s, in addition to reasonably low attenuation (1-2 dB/mm at 5 MHz). Several grades of castable urethane, such as Castall U-2941 from Lord Corp, also make suitable high velocity focusing lenses.

Alternatively, a focusing lens may incorporate a material with slower sound speed than water and tissue. The speed of sound is described with respect to water since the speed of sound in water is a known and well-established constant, and tissue is comprised to a large extent of water. RTV silicone rubbers, such as those from Dow Corning, are in this class. These materials can be matched to the acoustic impedance of tissue using higher density fillers such as aluminum oxide. They generally exhibit higher attenuation characteristics than the thermoplastics or urethanes previously described.

A flat, unfocused ultrasound transducer will generally emit plane waves, or waves characterized by planar phase fronts, having the same physical dimensions as the aperture in the very near field. Due to diffraction effects, the sound wave experiences a series of rapid fluctuations in intensity as it propagates into the tissue throughout the near field of the transducer before peaking at the natural focus and diverging beyond. The transition between the near and far field of the aperture occurs at a depth of roughly d²/3*λ, where d is the width of the aperture and λ is the sound wavelength in the medium.

For application of therapeutic ultrasound to superficial tissue, such as the dermis or subcutis, it is desirable to preferentially reduce the sound intensity beyond this region, while maintaining higher intensity in the superficial region.

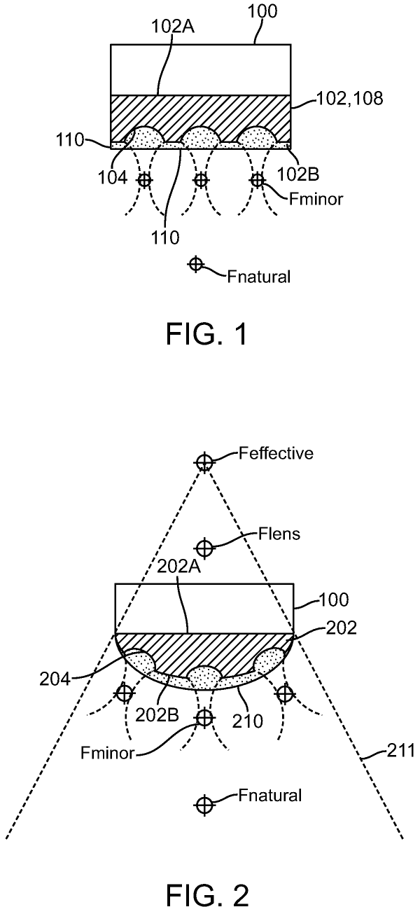

FIG. 1 is a sectional view of an ultrasound treatment apparatus including ultrasound transducer 100 and a multi-focal ultrasound lens 102 according to the present invention.

In each of the embodiments described in this specification, the lens has a first surface acoustically coupled to the transducer by means of direct mechanical bonding or using a coupling fluid, and a second surface which, in use, is acoustically coupled to the treatment zone, preferably by means of an acoustic coupling medium.

The flat transducer 100 has a natural focus Fₙₐₜᵤᵣₐₗ, whereas the lens 102, comprised of material with a sound speed higher than water, includes at least two and preferably plural minor lenses 104 each having their own geometric foci Fₘᵢₙₒᵣ. Importantly, Fₘᵢₙₒᵣ is less than (closer to the transducer 100) Fₙₐₜᵤᵣₐₗ. The minor focusing lenses 104 serve to decrease the effective aperture size and produce local high intensity regions in the vicinity of Fₘᵢₙₒᵣ that may result in a fractionated treatment pattern. According to one embodiment, Fₙₐₜᵤᵣₐₗ is between 10 mm and 50 mm, and Fₘᵢₙₒᵣ is between 1 mm and 20 mm.

The minor lenses 104 may be positioned on the surface 102A (the surface abutting the transducer) or surface 102B (the surface abutting the tissue undergoing treatment), and may be convex or concave in shape, depending on the material. In the illustrated embodiment, plural minor lenses 104 are positioned on the surface 102B.

To avoid repetition it should be understood that the ultrasound transducer disclosed in any of the embodiments described in this specification may be configured to produce sound pressures below the cavitation threshold of tissue and below the emulsification threshold of tissue. The ultrasound transducer produces ultrasound in which the predominant waveform is one of unfocused, focused, and defocused. According to a presently preferred embodiment, the transducer is configured to yield ultrasound having a mechanical index between 1.9 and 2.5, and the minor lenses of the multifocal lens increase the mechanical index within the treatment region to between 2 and 8.

It should further be understood that the minor lenses in any of the embodiments disclosed herein may be arranged in either a symmetric or a non-symmetric pattern. Moreover, a given lens may include minor lenses having different configurations. Thus the size and/or shape of selected ones of the plurality of minor lenses may be configured to create one of a different minor focus Fₘᵢₙₒᵣ or a different peak acoustic pressure in the treatment region.

FIG. 2 is a sectional view of an ultrasound apparatus including a diverging multi-focal ultrasound lens 202 according to the present invention. The diverging lens 202 is comprised of material with higher sound speed than water, thus the broad curvature of the lens surface 202B, with geometric focus Fₗₑₙₛ located behind the transducer 100, creates a diverging beam profile 211 when combined with the natural focus of the transducer, Fₙₐₜᵤᵣₐₗ (F_{effective}=(1/Fₙₐₜᵤᵣₐₗ+1/Fₗₑₙₛ)⁻¹). The minor lenses 204 may be positioned on the surface 202A (the surface abutting the transducer) or surface 202B (the surface abutting the tissue undergoing treatment), and may be convex or concave in shape, depending on the material. In the illustrated embodiment, plural minor lenses 204 are positioned on the surface 202B. The minor lenses 204 each have foci Fₘᵢₙₒᵣ that produce local high intensity regions in the near field. The combined effect of the lens 202 is a fractionated high pressure treatment pattern in the near field with rapidly decreasing pressure with depth.

Lenses 102 or 202 may optionally include a wear layer 110, 210 (shown shaded in FIGS. 1 and 2) that is selected to have different acoustic properties from the lens 102, 202. The wear layer 110, 210 provides a smooth surface to present to the tissue being treated. Moreover, depending on the acoustic properties of the material used, the wear layer 110, 210 may provide additional focusing. According to one embodiment, the lens 102, 202 has a faster speed of sound than the wear layer 110, 210 (cₗₑₙₛ>c_{wear}), and the wear layer has a sound speed equivalent to tissue. This causes the wear layer 110, 210 to act as a stand off. In another embodiment, the wear layer 110, 210 has sound speed lower than that of tissue (cₗₑₙₛ>cₜᵢₛₛᵤₑ>c_{wear}), which causes the wear layer to provide additional focusing (if convex) or defocusing (if concave). The wear layer 110, 210 is acoustically coupled to the lens surface 102B, 202B. The wear layer 110, 210 may, for example, be molded onto the lens surface 102B, 202B.

According to a presently preferred embodiment, the focal layer is formed of TPX and the wear layer 110, 210, if used, is formed of an RTV such as RTV 30, 60, 90 or similar.

The lens in each of the embodiments described in this specification may be removably connected to the transducer and may be a disposable component replaced independent of the transducer. This feature of the invention enables the user to adjust the focal depth simply by selecting a lens whose focal layer has the desired minor foci Fₘᵢₙₒᵣ. For example, the apparatus may be sold as a kit including a plurality of lenses (each having different minor foci Fₘᵢₙₒᵣ) thereby enabling the user to choose the desired treatment pattern.

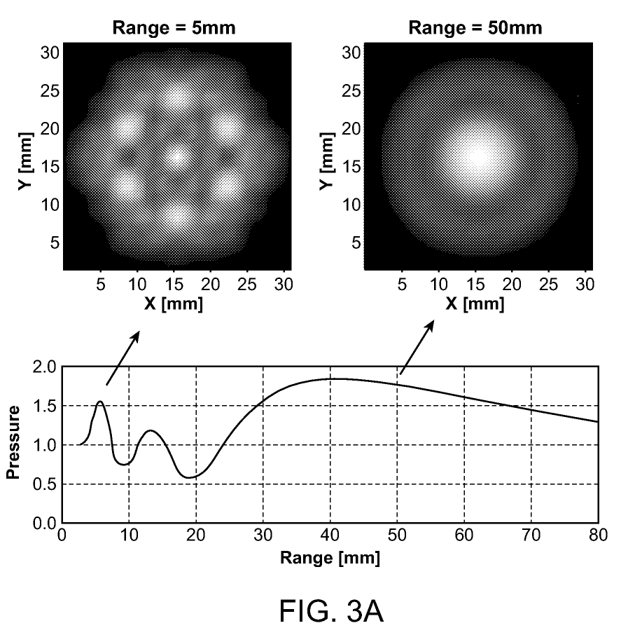

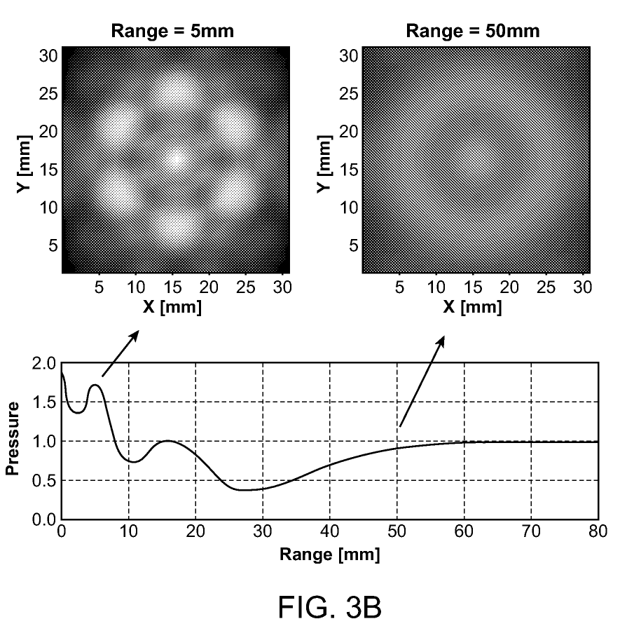

FIG. 3A depicts a simulation of on-axis (axis normal to the center of the transducer face) sound pressure for a flat 500 kHz transducer of 1 inch diameter of the type depicted in FIG. 1, and FIG. 3B depicts a simulation of the same transducer with a spherical 20 mm (diverging) lens of the type depicted in FIG. 2. Without the lens, the sound field exhibits fluctuations in near field before peaking at a distance of approximately 50 mm from the face of the transducer, corresponding to the near/far field transition. The lens 202 pushes out the near field peaks and rapidly reduces the pressure at depth, and in this case serves to concentrate the ultrasound energy in the region proximal to 20 mm. In cases where high pressure ultrasound is desired for superficial treatment, this technique may be employed to reduce pressures at depth to low levels, e.g., below diagnostic limits.

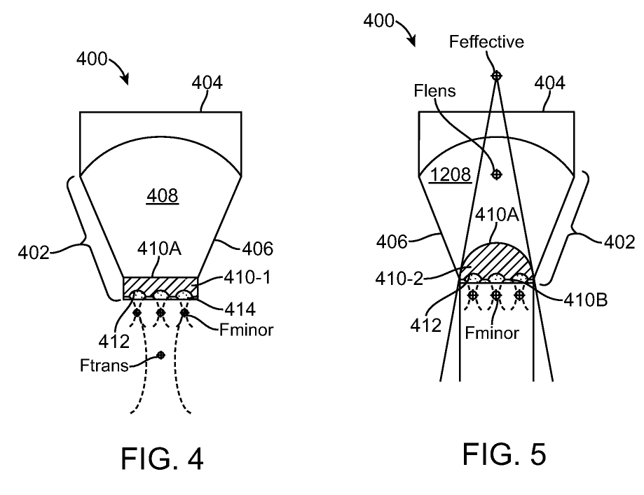

FIGS. 4 and 5 illustrate an ultrasound treatment apparatus 400 including a lens assembly 402 and a mechanically focused transducer 404 with focus Fₜᵣₐₙₛ. The lens assembly 402 includes a housing 406 and a lens 410-1 (FIG. 4) and 410-2 (FIG. 5) that include at least two and preferably plural minor lenses 412 each having their own geometric focus Fₘᵢₙₒᵣ. The housing 406 defines a hollow cavity 408. An acoustic coupling medium such as degassed water is provided within the cavity 408 to acoustically couple the transducer 404 with the lens 410-1, 410-2.

The portion of the lens through which ultrasound is transmitted and which surrounds the minor lenses 412 is referred herein as the “major lens” or the “major portion” of the lens. The major portion of the lens 410-1 (FIG. 4) is generally flat and smooth, and does not significantly contribute to focusing or defocusing of the beam produced by the mechanically focused transducer 404. In the embodiment illustrated in FIG. 4, the minor lenses 412 are primarily responsible for focusing of the ultrasound and create high pressure regions in the vicinity of Fₘᵢₙₒᵣ. It should be noted that the focus Fₘᵢₙₒᵣ of the minor lenses 412 is shallower than focus Fₜᵣₐₙₛ.

In contrast, the diverging lens 410-2 (FIG. 5) has a focus Fₗₑₙₛ that counteracts the transducer's mechanical focus and may be designed to provide a weakly focused beam, diverging beam or collimated beam. The minor lenses 412 may be positioned on the forward surface 410A or back surface 410B of the focal layer 410-1, 410-2. As with the flat transducer, the minor lenses 412 produce a pattern of local high intensity regions in the near field, while the major lens design limits the sound intensity at the focal length of the focused transducer.

An optional wear layer 414 (shown in dashed lines) that is selected to have different acoustic properties from the lens 410-1, 410-2 provides a smooth surface to present to the tissue being treated. Moreover, depending on the acoustic properties of the material used, the wear layer 414 may provide additional focusing.

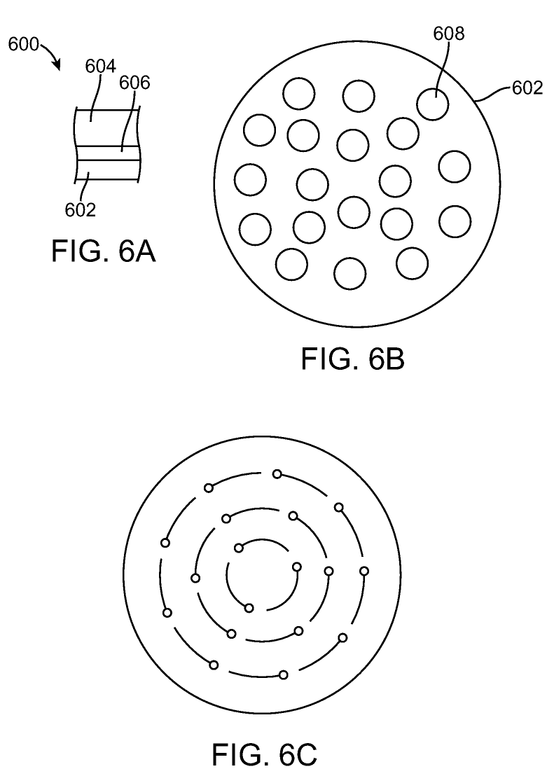

FIG. 6A is a functional diagram of an ultrasound apparatus 600 according to an alternate embodiment. The ultrasound apparatus 600 includes an acoustic lens assembly 602 acoustically and rotationally coupled to an ultrasound transducer 604 by rotary unit 606. The transducer 604 may be a flat (unfocused) or a focused transducer and may include either a single layer or multilayer piezoelectric transducer. Rotary unit 606 rotationally couples the acoustic lens assembly 602 to the ultrasound transducer 604 such that the lens assembly 602 rotates relative to the transducer 604. An acoustically transparent lubricant (e.g. degassed water, Krytox® grease (DuPont), castor oil or glycerin) may be provided between the transducer 604 and the lens assembly 602, and may serve to reduce friction between and/or acoustically couple the rotating lens 602 and the transducer 604.

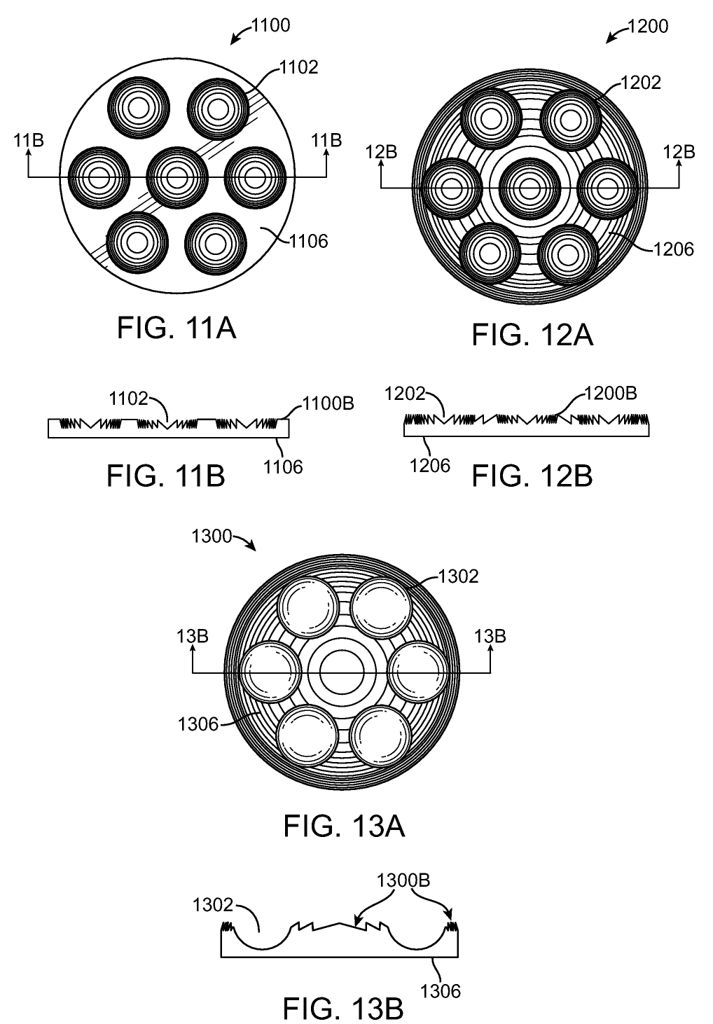

The lens assembly 602 may be any of the lens embodiments disclosed in this specification. In particular, the lens assembly 602 may include a lens 102 (FIG. 1), lens 202 (FIG. 2), lens 1102 (FIG. 11A), lens 1202 (FIG. 12A), or lens 1302 (FIG. 13A). The lens assembly 602 may also be lens assembly 402 (FIGS. 4 or 5). FIG. 6B is a bottom view of lens assembly 602 which includes plural dimple-like minor lenses 608. The rotary unit 606 may drive the lens assembly 602 in a generally circular path or may drive the lens in an elliptical path or the like. FIG. 6C shows the arc shaped treatment regions created by rotation of the lens assembly 602. Rotation of the lens assembly 602 effectively increases the treatment area without repositioning the handpiece.

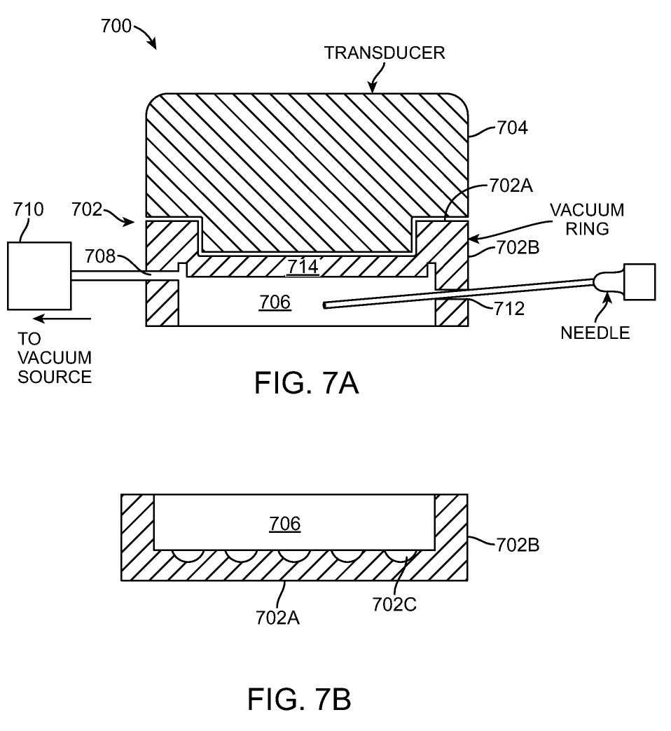

FIG. 7A is a sectional view of a tissue treatment apparatus 700 including a lens assembly 702 and transducer assembly 704. The lens assembly 702 includes a top surface 702A and downwardly depending sidewalls 702B defining a receptacle 706 for receiving tissue to be treated. In operation, the transducer 704 transmits ultrasound into the receptacle 706. The lens assembly 702 includes a lens 714. As best seen in FIG. 7B, the lens 714 optionally may include two or more minor lenses 702C. The lens assembly 702 may include a lens of the type illustrated in FIGS. 1, 2, 4, 5, 11-13B.

Turning once again to FIG. 7A, the lens assembly 702 may include a vacuum port 708 which is adapted to be fluidically connected to a source of reduced pressure 710, e.g., vacuum pump. Actuation of the vacuum pump 710 pulls tissue into the receptacle 706 and ensures a good acoustic coupling between the lens assembly 702 and the tissue.

The lens assembly 702 may optionally include a needle access port 712 providing access for injecting cavitation nuclei such as microbubbles or the like into the tissue undergoing treatment.

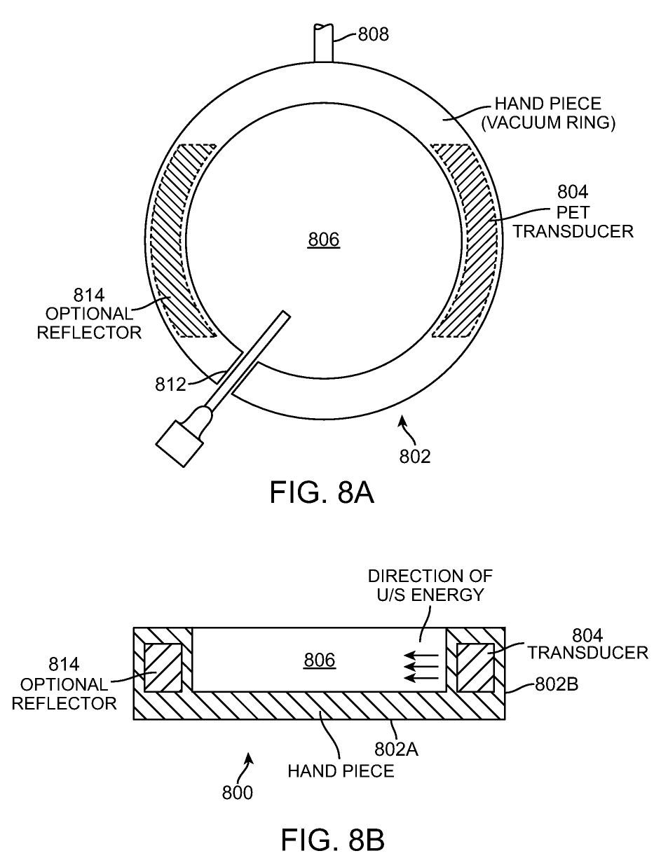

FIGS. 8A and 8B are bottom and sectional views of an ultrasound treatment device 800 including a lens assembly 802 and a side firing transducer 804. The lens assembly 802 includes a top surface 802A and downwardly depending sidewalls 802B which define a receptacle 806 for receiving tissue to be treated. The lens assembly 802 may optionally include a vacuum port 808 for fluidically coupling the receptacle 806 to a source of reduced pressure 810, e.g., a vacuum pump, and may include an optional needle access port 812. Lens assembly 802 is acoustically coupled to a transducer assembly 804 which directs ultrasound into the receptacle 806. In this embodiment, the transducer assembly 804 directs the ultrasound laterally through the side 802B of the lens assembly 802 into the receptacle 806. Also, in the embodiment shown in FIG. 8B the transducer 804 is molded into the sidewall 802B. However, the transducer 804 may also be provided on either the inner or outer surface of the sidewall 802B. In operation the ultrasound is directed though the tissue undergoing treatment (in the receptacle 806) and does not generally pass through the body thereby minimizing potential harm to tissue structures outside the receptacle 806.

In this embodiment, the sidewalls 802B form the acoustic lens whereas in the embodiment shown in FIG. 7A the top surface 702A forms the acoustic lens. The acoustic lens assembly 802 may include a single lens or may include a plurality of minor lenses (not illustrated) on the sidewalls 802B. The minor lenses may have any of the geometries disclosed in this application. Namely, the minor lenses may be concave, convex, or as will be described below, Fresnel.

The lens assembly 802 may optionally include an acoustic reflector 814 which may be molded into sidewall 802B or may be attached to the interior or exterior sides of sidewall 802B. The acoustic reflector 814 is preferably provided in a facing relationship with the ultrasound transducer 804. If desired the device 800 may include two or more transducers 804 with an equal number of acoustic reflectors 814 provided in a facing relationship (not illustrated). Transducer 804 may be molded into sidewall 802B or may be provided on the interior or exterior sides of sidewall 802B.

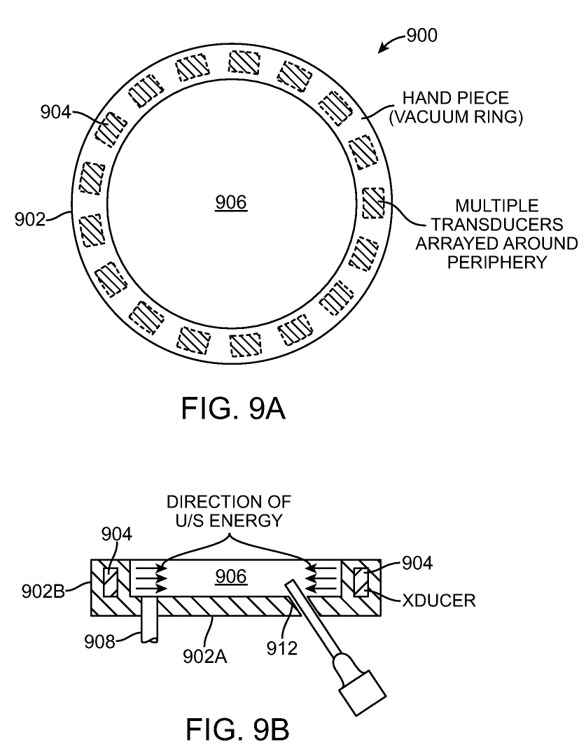

FIGS. 9A and 9B illustrate an ultrasound treatment device 900 including a lens assembly 902 and a plurality of transducers 904. The lens assembly 902 includes a top surface 902A and downwardly depending sidewalls 902B which define a receptacle 906. The transducers 904 ring or encircle the receptacle 906 and may be molded into the sidewalls 902B or attached to the interior (within the receptacle 906) or exterior surfaces of the side wall 902B. Like the embodiments shown in FIGS. 7A-7B, the assembly 902 may optionally include a vacuum port 908 for fluidically coupling the receptacle 906 with a vacuum pump (not illustrated), and/or may include a needle access port 912 for inserting a needle into the tissue undergoing treatment. The transducers 904 may be operated in a time staggered manner.

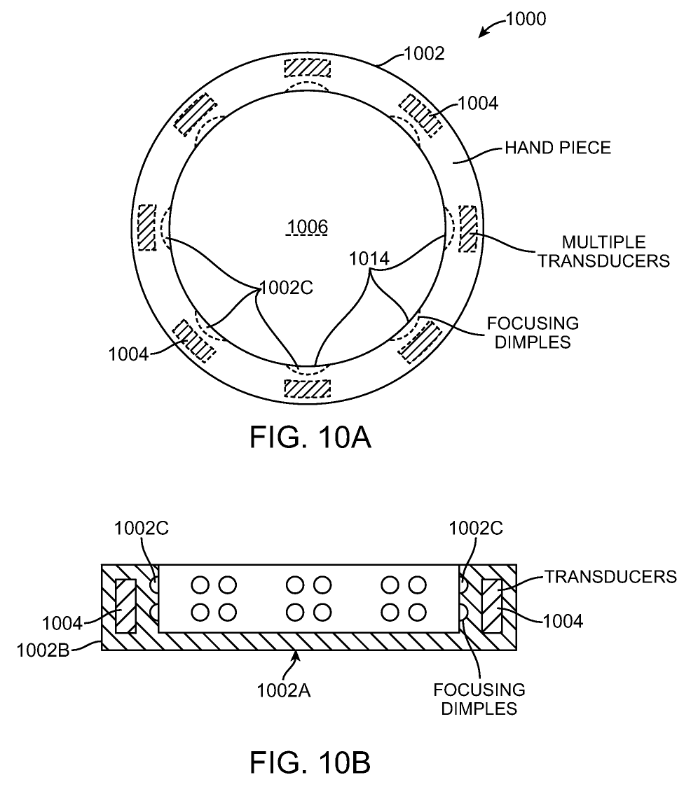

FIGS. 10A and 10B show an ultrasound treatment system 1000 including lens assembly 1002 which defines a receptacle 1006 configured to receive tissue undergoing treatment. The receptacle 1006 is defined by sidewalls 1002B and top surface 1002A. Plural transducers 1004 encircle or ring the receptacle 1006 and are either provided on the sidewalls 1002B or are molded into the sidewalls 1002B. The sidewalls 1002B define an acoustic lens. Two or more minor lenses 1002C of the type described elsewhere in this application are provided on the sidewall 1002B in opposition to the transducers 1004.

The lens assembly 1002 may optionally include a vacuum port (not shown) for fluidically coupling the receptacle 1006 to a source of reduced pressure (not shown), e.g., a vacuum pump, and may include an optional needle access port (not shown).

The minor lenses 1002C may be positioned on the inner surface of the sidewall 1002B or on the surface abutting the transducer 1004 and may be a converging lens or a diverging lens. Moreover, the lens assembly 1002 may be provided with a wear layer 1014 (shown in dashed lines) to present a smooth surface to the tissue undergoing treatment.

FIG. 11 depicts an alternate embodiment of lens 102 (FIG. 1). FIGS. 11A and 11B show a lens 1100 including at least one and preferably plural minor lenses 1102, each minor lens 1102 being a focusing Fresnel lens formed (molded or machined) into surface 1100B. The Fresnel lens ring spacing may follow the approach described by Fjield et al., Low-profile lenses for ultrasound surgery, PHYS. MED. BIOL. 44 (1999). FIG. 11B is a sectional view of FIG. 11A along line 11B-11B. In the illustrated embodiment, the major lens 1106 is generally flat and smooth, and does not significantly contribute to the focusing or defocusing of the lens 1100. In other words, most of the focusing is performed by the minor lenses 1102. If additional focusing is desired, the shape of the major lens 1106 may be modified to provide additional focusing.

FIGS. 12-13 depict an alternate embodiment of lens 202 (FIG. 2). FIGS. 12A-12B show a lens 1200 including a major lens 1206 and two or more minor focusing Fresnel lenses 1202 formed on the surface 1200B. FIG. 12B is a sectional view of FIG. 12A along line 12B-12B. The major lens 1206 is comprised of a diverging Fresnel lens which serves to defocus the broad ultrasound beam, while the minor focusing Fresnel lenses 1202 serve to increase the local pressure in the region of the minor lens foci.

FIGS. 13A-13B show a lens 1300 including a major lens 1306 and two or more spherical minor focusing lenses 1302 formed on or machined into the surface 1300B. FIG. 13B is a sectional view of FIG. 13A along line 13B-13B. As in FIG. 12, the major lens 1306 is a diverging Fresnel lens and contributes to the defocusing of the broad ultrasound beam. As with minor lenses 1102 and 1202, the spherical minor lenses 1302 serve to increase the local pressure in the region of the minor lens foci.

Lenses 1100, 1200 and 1300 can provide comparable ultrasound focusing characteristics to those lenses shown in FIGS. 1 and 2 while providing lower profiles. Thinning the lens offers a means to reduce sound attenuation and improve performance while reducing overall device dimensions.

The invention may be embodied in other forms without departure from the spirit and essential characteristics thereof The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

We claim:

1. An ultrasound apparatus comprising:
an ultrasound transducer having a geometric focus Fₜᵣₐₙₛ, said transducer producing ultrasound waves; and
an acoustic lens assembly including a focal layer acoustically coupled to the ultrasound transducer such that said ultrasound waves are directed through the focal layer, said focal layer including at least two minor lenses, each said minor lens having a geometric focus Fₘᵢₙₒᵣ, each said minor lens yielding a discrete treatment region, such that the focal layer includes a plurality of discrete treatment regions.

2. The ultrasound apparatus of claim 1, wherein the ultrasound transducer produces ultrasound having a mechanical index between 1.9 and 2.5, and each said minor lens increases the mechanical index within the treatment region to between 2 and 8.

3. The apparatus of claim 1, wherein the minor lenses have a shape selected from the group consisting of concave, Fresnel concave, convex and Fresnel convex.

4. The apparatus of claim 1, wherein the minor lenses are arranged in a non-symmetric pattern.

5. The apparatus of claim 1, wherein a size and shape of selected ones of the plurality of minor lenses are configured to create one of a different geometric focus Fₘᵢₙₒᵣ or a different peak acoustic pressure in the treatment region.

6. The apparatus of claim 1, wherein the focal layer includes major lens portions interspersed between the minor lenses, and wherein the major lens portions have a shape selected from the group of flat, Fresnel, concave, Fresnel concave, convex, and Fresnel convex.

7. The apparatus of claim 1, wherein the transducer produces one of unfocused, defocused, and focused ultrasound.

8. The apparatus of claim 1, wherein the transducer is removably connected to the acoustic lens.

9. The apparatus of claim 1, further comprising a wear layer operably connected to the focal layer and having different acoustic properties from the focal layer.

10. The apparatus of claim 9, wherein the focal layer has a different speed of sound than tissue and the wear layer.

11. The apparatus of claim 1, further comprising a rotational drive mechanism operably attached to the lens assembly, said drive mechanism rotationally driving the lens assembly such that the lens assembly rotates relative to the transducer.

12. The apparatus of claim 1, wherein the transducer is one of flat, concave, and convex.

13. The apparatus of claim 1, wherein the transducer produces ultrasound in which the predominant waveform is one of unfocused, focused, and defocused.

14. An ultrasound apparatus comprising:
an ultrasound transducer having a geometric focus Fₜᵣₐₙₛ; and
a diverging acoustic lens assembly having a focal layer, said diverging lens assembly acoustically coupled to the ultrasound transducer, said focal layer including at least two minor lenses, each said minor lens having a geometric focus Fₘᵢₙₒᵣ, each said minor lens yielding a discrete treatment region such that the focal layer includes plural discrete treatment regions, said focal layer having a geometric focus Fₗₑₙₛ where Fₗₑₙₛ<0, and 0<Fₘᵢₙₒᵣ<Fₜᵣₐₙₛ, wherein the effective geometric focus of the transducer with the diverging acoustic lens is F_{effective}, and wherein F_{effective}<0.

15. The ultrasound apparatus of claim 14, wherein the ultrasound transducer produces ultrasound having a mechanical index between 1.9 and 2.5, and the focal layer increases the mechanical index within the treatment region to between 2 and 8.

16. The apparatus of claim 14, wherein the minor lenses have a shape selected from the group consisting of concave, Fresnel concave, convex and Fresnel convex.

17. The apparatus of claim 14, wherein the minor lenses are arranged in a non-symmetric pattern.

18. The apparatus of claim 14, wherein a size and shape of a selected one of the at least two minor lenses is configured to create one of a different geometric focus Fₘᵢₙₒᵣ or a different peak acoustic pressure in the discrete treatment region.

19. The apparatus of claim 14, wherein the focal layer includes major lens portions interspersed between the minor lenses, wherein the major lens portions have a shape selected from the group of flat, Fresnel, concave, Fresnel concave, convex, and Fresnel convex.

20. The apparatus of claim 14, wherein the transducer produces an ultrasound having a predominant waveform being one of unfocused, defocused, or focused.

21. The apparatus of claim 14, wherein the transducer is removably connected to the acoustic lens.

22. The apparatus of claim 14, further comprising a wear layer operably connected to the focal layer and having different acoustic properties from the focal layer.

23. The apparatus of claim 22, wherein the focal layer has different speed of sound than tissue and the wear layer.

24. The apparatus of claim 14, further comprising a rotational drive mechanism operably attached to the lens assembly, said drive mechanism rotationally driving the lens assembly such that the lens assembly rotates relative to the transducer.

25. An ultrasound apparatus comprising:
a focused ultrasound transducer having a geometric focus Fₜᵣₐₙₛ; and
an acoustic lens assembly having a focal layer, said lens assembly acoustically coupled to the ultrasound transducer, said focal layer including at least two minor lenses, each said minor lens having a geometric focus Fₘᵢₙₒᵣ, each said minor lens yielding a discrete treatment region, such that the focal layer includes plural discrete treatment regions, said focal layer having a geometric focus Fₗₑₙₛ where 0<Fₘᵢₙₒᵣ<Fₜᵣₐₙₛ, and the effective geometric focus of the transducer with the diverging acoustic lens is F_{effective} where F_{effective}<0.

26. The ultrasound apparatus of claim 25, wherein the ultrasound transducer produces ultrasound having a mechanical index between 1.9 and 2.5, and the focal layer increases the mechanical index within the treatment region to between 2 and 8.

27. The apparatus of claim 25, wherein the minor lenses have a shape selected from the group consisting of concave, Fresnel concave, convex and Fresnel convex.

28. The apparatus of claim 25, wherein the minor lenses are arranged in a non-symmetric pattern.

29. The apparatus of claim 25, wherein a size and shape of selected ones of the plurality of minor lenses are configured to create one of a different geometric focus Fₘᵢₙₒᵣ or a different peak acoustic pressure in the discrete treatment region.

30. The apparatus of claim 25, wherein the focal layer includes major lens portions interspersed between the minor lenses, and wherein the major lens portions have a shape selected from the group of flat, Fresnel, concave, Fresnel concave, convex, and Fresnel convex.

31. The apparatus of claim 25, wherein the transducer produces an ultrasound having a predominant waveform being one of unfocused, defocused, or focused.

32. An ultrasound apparatus comprising:
a focused ultrasound transducer having a geometric focus Fₜᵣₐₙₛ; and
a diverging acoustic lens assembly having a focal layer, said diverging lens assembly acoustically coupled to the ultrasound transducer, said focal layer including at least two minor lenses, each said minor lens having a geometric focus Fₘᵢₙₒᵣ, each said minor lens yielding a discrete treatment region, such that the focal layer includes plural discrete treatment regions, said focal layer having a geometric focus Fₗₑₙₛ<0, where 0<Fₘᵢₙₒᵣ<Fₜᵣₐₙₛ, wherein the effective geometric focus of the transducer with the diverging acoustic lens is F_{effective}, and wherein F_{effective}<0.

33. The ultrasound apparatus of claim 32, wherein the ultrasound transducer produces ultrasound having a mechanical index between 1.9 and 2.5, and the focal layer increases the mechanical index within the treatment region to between 2 and 8.

34. The apparatus of claim 32, wherein the minor lenses have a shape selected from the group consisting of concave, Fresnel concave, convex and Fresnel convex.

35. The apparatus of claim 32, wherein the minor lenses are arranged in a non-symmetric pattern.

36. The apparatus of claim 32, wherein a size and shape of selected ones of the plurality of minor lenses are configured to create one of a different geometric focus Fₘᵢₙₒᵣ or a different peak acoustic pressure in the treatment region.

37. The apparatus of claim 32, wherein the focal layer includes major lens portions interspersed between the minor lenses, where the major lens portions have a shape selected from the group of flat, Fresnel, concave, Fresnel concave, convex, and Fresnel convex.

38. The apparatus of claim 32, wherein the transducer produces ultrasound in which the predominant waveform is one of unfocused, defocused, and focused.

39. The apparatus of claim 32, wherein said acoustic lens assembly includes a receptacle for receiving tissue to be treated, said receptacle bounded on top and encircled by downwardly depending sidewalls, said transducer directing ultrasound into the receptacle.

40. The apparatus of claim 32, wherein the acoustic lens assembly includes at least one of a vacuum port and a needle access port.

41. The apparatus of claim 40 further comprising a source of suction fluidically coupled with said vacuum port.

42. The apparatus of claim 39 wherein the focal layer forms the top wall of the receptacle.

43. The apparatus of claim 39 wherein the focal layer forms the downwardly depending sidewalls of the receptacle.

44. The apparatus of claim 43, further comprising an ultrasound reflector operably connected to the downwardly depending sidewalls such that the reflector generally faces the ultrasound transducer, whereby said reflector reflects ultrasound back into the receptacle.

45. The apparatus of claim 39, wherein said ultrasound transducer comprises a plurality of ultrasound transducers surrounding the receptacle.

46. The apparatus of claim 45, further comprising a plurality of ultrasound reflectors operably connected to the downwardly depending sidewalls such that each reflector generally faces an ultrasound transducer, whereby said reflectors reflects ultrasound back into the receptacle.

47. The apparatus of claim 45 wherein the plurality of transducers are molded into the sidewalls.

48. The apparatus of claim 45 wherein the plurality of transducers are attached to an interior and within the receptacle.

49. The apparatus of claim 45 wherein the plurality of transducers are attached to an exterior surface of the sidewalls.

50. The apparatus of claim 45 wherein the plurality of transducers are operated in a time staggered manner.

51. An ultrasound apparatus comprising:
an ultrasound transducer having a geometric focus Fₜᵣₐₙₛ, said transducer producing ultrasound waves; and
an acoustic lens assembly including a focal layer acoustically coupled to the ultrasound transducer such that said ultrasound waves are directed through the focal layer,
said acoustic lens assembly defining a receptacle for receiving tissue to be treated, said receptacle bounded on top and encircled by downwardly depending sidewalls, said transducer directing ultrasound into the receptacle.

52. The apparatus of claim 51, wherein the acoustic lens assembly includes at least one of a vacuum port and a needle access port.

53. The apparatus of claim 52 further comprising a source of suction fluidically coupled with said vacuum port.

54. The apparatus of claim 51 wherein the focal layer forms the top wall of the receptacle.

55. The apparatus of claim 51 wherein the focal layer forms the downwardly depending sidewalls of the receptacle.

56. The apparatus of claim 51, wherein said ultrasound transducer comprises a plurality of ultrasound transducers surrounding the receptacle.

57. The apparatus of claim 56 wherein the plurality of transducers are molded into the sidewalls.

58. The apparatus of claim 56 wherein the plurality of transducers are attached to an interior and within the receptacle.

59. The apparatus of claim 56 wherein the plurality of transducers are attached to an exterior surface of the sidewalls.

60. The apparatus of claim 56 wherein the plurality of transducers are operated in a time staggered manner.
